(19) United States
(12) Patent Application Publication
(10) Pub. No.: US 2008/0176320 A1
(43) Pub. Date: Jul. 24, 2008

(54) Selective Lysis of Sperm Cells

(75) Inventor: Yingjie Liu, Foster City, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 12/015,414

(22) Filed: Jan. 16, 2008

Related U.S. Application Data
(60) Provisional application No. 60/880,787, filed on Jan. 16, 2007.
(60) Provisional application No. 60/899,106, filed on Feb. 2, 2007.

(51) Int. Cl. C12N 5/06

(52) U.S. Cl. 435/325

(57) ABSTRACT

The teaching provides a method for selectively lysing sperm cells in a mixed cell sample, particularly in the field of forensic sciences. Reagents and kits for carrying out the methods are also provided.

### CROSS REFERENCE TO RELATED APPLICATIONS

This application claims a priority benefit under 35 U.S.C. §119(e) to U.S. Application No. 60/880,787 filed Jan. 16, 2007 and U.S. Application No. 60/899,106 filed Feb. 2, 2007, the entire contents of each is incorporated herein by reference.

### FIELD

The teaching is in the area of selective extraction of DNA from groups of cells. Selective lysis of a particular cell type within a cellular mixture is performed and then the mixture is separated with a mean that allows the DNA from the lysed cells to be separated from unlysed cells, thereby selectively extracting the DNA from a particular cell type.

### INTRODUCTION

Forensic DNA analysis of sexual assault evidence often involves analysis of DNA from sperm cells and DNA from other cells such as epithelial cells. The samples obtained from victims often contain a mixture of sperm and other cells such as epithelial cells. Because other cells such as epithelial cells may outnumber sperm cells by many folds, it may cause contamination from other source of DNA while sperm DNA is extracted. Therefore, it is often desirable to separate the sperm cells and epithelial cells, or the sperm DNA and the epithelial DNA as clean as possible, prior to analysis Separation and isolation of DNA from sperm and epithelial cells to create an accurate profile are critical for identification of an assailant.

Differential extraction is a broad term used to describe several extraction methods that can be used to separate cells. Unique characteristics of sperm cells allow for the differential extraction of the epithelial cells from the sperm cells. The first differential extraction procedure was described in 1985 (Gill et al. (1985) Nature 318: 557-9). Separation of the male fraction from the victim's DNA profile removes ambiguity in the results and allows for easier interpretation of the perpetrator's DNA profile in a rape case. Although differential extraction is commonly used to separate sperm and epithelial cells, the standard protocol is a time consuming and laborious process.

The differential extraction procedure involves preferentially breaking open the female epithelial cells with an incubation in a sarkosyl/proteinase K mixture. Sperm cells are subsequently lysed by treatment with a sarkosyl/proteinase K/dithiothreitol (DTT) mixture. The DTT breaks down the protein disulfide bridges that make up sperm nuclear membranes (Gill et al. (1985) Nature 318: 557-9). Differential extraction is effective because sperm cells are strengthened with cross-linked thiol-rich proteins, which render them impervious to digestion without DTT. However, this method is cumbersome in procedure and may cause the loss of the sperm DNA due to lengthy and repetitive sample handling.

Therefore, creating a reliable, accurate profile of sperm DNA is imperative for human identification in forensic science.

### SUMMARY

The current teaching provides an alternative method for achieving differential extraction. Some of the advantages of the current teaching are reduced sample process time, simplified work flow and the ability to process solid substrates (such as swab) directly. Moreover the teaching provides a method for obtaining an increasing amount of sperm DNA when the sample is scarce; therefore the detection sensitivity is increased. This selective sperm DNA extraction assay is applicable to any sample which contains sperm cells or sperm cell mixed with other multiple kinds of cells containing DNA, and the DNA can be of human (including animal) or plant origin or any combination of human, animal or plant DNA.

The teaching provides a selective sperm lysis buffer comprises at least one disulfide bond reducing reagent and at least one salt reagent. wherein the concentration of the salt reagent is at least 0.1M, 0.5M, 1M, 2M or higher.

In some embodiment, the selective sperm lysis buffer consists essentially of or consists of at least one disulfide bond reducing reagent and at least one salt reagent. wherein the concentration of the salt reagent is at least 0.1M, 0.5M, 1M, 2M or higher.

The salt reagent in the teaching is selected from the group comprising LiCl, NaCl, KCl. Li₂SO₄, Na₂SO₄, K₂SO₄, MgCl₂, CaCl₂, MgSO₄, CaSO₄, NaNO₃, KNO₃, Mg(NO₃)₂, and Ca(NO₃)₂.

In certain embodiment, the salt comprises NaCl, KCl or MgCl₂,

The disulfide bond reducing reagent of the teaching comprises at least DTT, TCEP, ME or GSH.

The teaching further provides a method of selectively lysing a sperm cell in a mixture of at least one type of non-sperm cell comprises i) incubating the mixture with the selective sperm lysis buffer, wherein the buffer comprises at least one disulfide bond reducing reagent and at least one salt reagent, wherein the salt reagent is at a concentration to lyse a sperm cell whereas a non-sperm cell is intact, ii) lysing the sperm cell, while the non-sperm cell is intact; and iii) separating the non-sperm cell from the lysed sperm cell. Alternatively the buffer can be used in a concentration described above.

One skilled in the art can determine the concentration of the salt reagent to ensure the lysis of the sperm cell while to keep the other non-sperm cell intact.

The non-sperm cells in the mixture of the teaching is selected from the group including, but not limited to, erythrocytes, platelets, neutrophils, lymphocytes, monocytes, eosinophils, basophils, adipocytes, chondrocytes, tumor cells, neurons, glial cells, astrocytes, red blood cells, white blood cells, macrophages, hair cells, bladder cells, kidney cells, retinal cells, rod cells, cone cells, antigen presenting cells, T cells, B cells, plasma cells, muscle cells, ovarian cells, prostate cells, vaginal epithelial cells, testicular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, osteociasts,

The teaching further provides a method of differential extraction of sperm cell DNA from a cell mixture from a sample carrier, wherein the cell mixture comprises a sperm cell and at least one type of non-sperm cell, comprises: i) washing the cell mixture with washing buffer and remove the supernatant, ii) incubating the mixture with the selective sperm lysis buffer, wherein the buffer comprises at least on disulfide bond reducing reagent and at least one salt reagent, wherein the salt reagent is at a concentration to lyse a sperm cell whereas a non-sperm cell is intact, iii)lysing the sperm cell, while the non-sperm cell is intact, and iv)separating the non-sperm cell from the lysed sperm cell.

The teaching further provides a kit for isolating sperm cell DNA from non-sperm cell DNA comprises (i) a selective sperm lysis buffer comprising at least one disulfide bond reducing agent and at least one salt, wherein the salt is at the concentration wherein the sperm cell is lysed while the other cells are intact.

The kit of the teaching can be combined or integrated into other applications comprising DNA purification.

### DRAWINGS

The skilled artisan will understand that the drawings, described below, are for illustration purposes only. The drawings are not intended to limit the scope of the present teachings in any way.

FIG. 1: a. Fluorescent image of unlysed epithelial cell and sperms in sample 1. b. Fluorescent image of epithelial cell and sperms after selective sperms lysis in sample 2. Sperms were completely lysed and only sperm tails were left. Epithelial cell is still intact.

FIG. 2: STR profiles in green channel from sperm fraction.

FIG. 3: STR profiles in blue channel from sperm fraction.

FIG. 4: Top panel. STR profiles in green channel from sperm fraction. Bottom panel, STR profiles in green channel from epithelial fraction

FIG. 5: STR profiles in blue channel from sperm fraction.

### DESCRIPTION OF VARIOUS EMBODIMENTS

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in this application, this application controls.

The use of the singular includes the plural unless specifically stated otherwise. The word “a” or “an” means “at least one” unless specifically stated otherwise. The use of “or” means “and/or” unless stated otherwise. The use of “or” in the context of multiply dependent claims means the alternative only. The meaning of the phrase “at least one” is equivalent to the meaning of the phrase “one or more.” Furthermore, the use of the term “including,” as well as other forms, such as “includes” and “included,” is not limiting. Also, terms such as “element” or “component” encompass both elements or components comprising one unit and elements or components that comprise more than one unit unless specifically stated otherwise.

In this specification, discussion of detecting “a” moiety, such as a target analyte, encompasses one or more of that moiety unless specifically stated otherwise. All ranges discussed herein include the endpoints and all values between the endpoints.

### Definitions

The term “biological sample” refers to any specimen that contains biological material.

The term “cell mixture” refers to a heterogeneous collection of at least two or more different cell types.

The term “differential extraction” refers to extraction methods utilized to separate cells within a heterogeneous population of cells, for example, the selective lysis of sperm cells in an epithelial-sperm cell mixture.

The term “disulfide bond reducing agent” as used herein, refers to an agent that disrupts protamine disulfide bridges in sperm cells. Disulfide bond reducing agents can be water-insoluble or water soluble agents. Exemplary water-insoluble agents include, but are not limited to dithiothreitol (DTT) and Tris(2-carboxyethyl)phosphine hydrochloride (TCEP). Exemplary water-soluble agents include, but are not limited to, glutathione (GSH) and mercaptoethanol (ME).

The term “forensic sample” refers to a sample obtained for use to address legal issues, including, but not limited to murder, rape, trauma, assault, battery, theft, burglary, other criminal matters, identity, parental or paternity testing, and mixed-up samples. It broadly refers to a material which contains biological materials such as blood, blood stains, saliva, saliva stains, skin debris, feces, feces stains, urine, sperm cells, vaginal epithelial cells, sperm epithelial cells, other epithelial cells, muscles, bone or muscle remains or mummified remains.

The term “lysate” as used herein, refers to a liquid phase with lysed cell debris and DNA.

The term “medical sample” refers to a sample obtained to address medical issues including, but not limited to research, diagnosis, or tissue and organ transplants.

The term “salt” or “salt reagent” or “salt solution” as used herein, refers to positively and/or negatively charged ionic reagent, which disrupting the sperm chromatin. The salt further refers to cation or anion which includes monovalent, biovalent or multivalent ion and family thereof in a solution. Exemplary salt include, but are not limited to LiCl, NaCl, KCl. Li₂SO₄, Na₂SO₄, K₂SO₄, MgCl₂, CaCl₂, MgSO₄ and CaSO₄, NaNO₃, KNO₃, Mg(NO₃)₂, Ca(NO₃)₂.

The term “sample carrier” as used herein, refers to a carrier that has forensic samples attached. The carrier can be, but is not limited to, clothing, swab, container, paper.

The term “selective sperm lysis buffer” as used herein, refers to a buffer that is capable of preferentially lysing sperm cells in a mixture comprising sperm cells and at least one type of non-sperm cells. “Preferentially lysing sperm cells” as used herein and thereafter, refers to that of sperm cells are lysed, whereas nonsperm cells are not lysed. Quantitatively, at least 80%, 85%, 90%, 95%, 99% of the sperm cells are lysed, whereas at least 80%, 85%, 90%, 95% of the nonsperm cells are not lysed. In certain embodiments, a negligible amount of non-sperm cells are lysed.

Optionally, the reagent in the buffer herein and thereafter can be mixed with cell mixture in a sequential manner. Optionally, the disulfide bond reducing agent is added in the mixture first and subsequently the salt reagent or salt solution is added.

The term “selective epithelial lysis buffer” as used herein, refers to a buffer that is capable of preferentially lysing epithelial cells in a mixture comprising sperm cells and epithelial cells. Preferentially lysing epithelial cells” means that of epithelial cells are lysed. In certain embodiments, a negligible amount of sperm cells are lysed.

The term “short tandem repeat” (STR) refers to all sequences between 2 and 7 nucleotides long which are tandemly reiterated within the human organism. The term “non-sperm cell” as used herein, refers to a cell that is not a sperm cell. In various embodiments, non-sperm cells can comprise one or more cell types. Exemplary non-sperm cells include, but are not limited to, epithelial cells, blood cells with all types, hair cells, or any other cells types can present in forensic samples.

The term “washing solution” used herein, refers to a reagent with a function of removing extraneous DNA from cells. Washing solution can be, but is not limited to water, PBS, Tris-HCl or other physiologically acceptable carrier.

### Certain Exemplary Selective Sperm Lysis Buffers

In certain embodiments, a selective sperm lysis buffer comprises at least one disulfide bond reducing reagent and at least one salt reagent

In certain embodiment, a disulfide bond reducing agent selected from reagents including, but not limited to, ME, DTT, GSH and TCEP

In certain embodiments, at least one salt is selected from reagents including but not limited to, LiCl, NaCl, KCl. Li₂SO₄, Na₂SO₄, K₂SO₄, MgCl₂, CaCl₂, MgSO₄ and CaSO₄, NaNO₃, KNO₃, Mg(NO₃)₂, Ca(NO₃)₂, In certain embodiment, at least one salt is NaCl, KCl, or MgCl₂.

In certain embodiment, a selective sperm lysis buffer comprises at least one salt reagent in the concentration at least 0.1M, 0.25M, 0.5M, 1M, 15M, 2M or higher.

In certain embodiment, a selective sperm lysis buffer comprises at least one salt reagent in the concentration range from 0.1M to 2M.

In certain embodiments, a selective sperm lysis buffer comprises at least one disulfide bond reducing reagent with concentration ranges from at least 0.01M, 0.05M, 0.1M, 0.2M, 0.3M, 0.4M, 0.5M, 0.7M or 0.8M.

In some embodiments, a selective sperm lysis buffer comprises disulfide bond reducing reagent selected from the group consisting of ME, DTT, GSH and TCEP and salt reagent selected from the group consisting of NaCl, KCl, MgCl₂ and CaCl₂, wherein the salt concentration is at the concentration to lyse the sperm cell and keep the non-sperm cell intact.

In some embodiment, NaCl or KCl concentration is at least 0.8M or higher, and MgCl₂ concentration is at least 0.25M or higher.

In certain embodiments, selective sperm lysis buffer comprises two reagents which when used in sequential manner can result in selective lysis of sperms in a mixture comprising sperm cells and at least one type of non-sperm cells. The first reagent comprises at least one disulfide bond reducing reagent with concentration ranges from at least 0.01M, 0.05M, 0.1M, 0.2M, 0.3M, 0.4M, 0.5M, 0.7M or 0.8M. The second reagent comprises at least one salt with concentration ranges from at least 0.1M, 0.25M, 0.5M , 1M, 1.5M, 2M or higher.

Based on the teaching, one of the skill in the art can optimize the final salt concentration level to breakdown the sperm cell while keep the non-sperm cell intact.

### Certain Exemplary Methods of Selective Lysis of Sperm Cells

In certain embodiment, a sample comprises at least two cell types is placed within a container which contains washing solution and the cells. Cells are dissociated from sample carrier such as swab, container or paper by means of washing solution. The washing solution includes, but is not limited to, water, PBS, TrisHCl or other physiologically acceptable carrier.

In certain embodiment, a sample comprises at least sperm cells.

Optionally, the container can be a tube, or a well, which is open on the top, and enclosed on all sides and the bottom. These wells can be joined together to form a plate. Multiple wells can be joined together to form a plate such as 96 well plate. Optionally, the well can contain a size exclusion filter, which is suspended and allows for an open space both above and below the filter and can be removable.

The samples can be from any source, for example, they can be biological, medical or forensic samples, including but not limited to the group consisting of cell culture, blood, semen, vaginal swabs, tissue, hair, saliva, urine, semen samples from rape victims, or semen samples from soiled clothing, identification of human remains, or any mixture of the preceding list or any mixture of body fluids or liquid form.

In some embodiment, the biological, medical or forensic sample is from a human, animal or plant. In some embodiment, the sample is a vaginal or oral swab obtained from a rape victim.

In certain embodiments, the sample are mixed with a selective sperm lysis comprises at least one disulfide bond reducing reagent and at least one salt reagent.

In certain embodiment, a disulfide bond reducing agent selected from reagents including, but not limited to, ME, DTT, GSH and TCEP

In certain embodiments, at least one salt is selected from reagents including but not limited to, LiCl, NaCl, KCl. Li₂SO₄, Na₂SO₄, K₂SO₄, MgCl₂, CaCl₂, MgSO₄ and CaSO₄, NaNO₃, KNO₃, Mg(NO₃)₂, Ca(NO₃)₂. In certain embodiment at least one salt is NaCl, KCl, or MgCl₂.

In certain embodiment, a selective sperm lysis buffer comprises at least one salt reagent in the concentration at least 0.1M, 0.25M, 0.5M, 1M, 1.5M, 2M or higher.

In certain embodiment, a selective sperm lysis buffer comprises at least one salt reagent in the concentration range from 0.1M to 2M.

In certain embodiments, a selective sperm lysis buffer comprises at least one disulfide bond reducing reagent with concentration ranges from at least 0.01M, 0.05M, 0.1M, 0.2M, 0.3M, 0.4M, 0.5M, 0.7M or 0.8M.

In certain embodiments, a selective sperm lysis buffer comprises disulfide bond reagent selected from the group consisting of ME, DTT, GSH and TCEP and salt reagent selected from the group consisting of NaCl, KCl, MgCl₂ and CaCl₂, wherein the salt concentration is at the concentration to lyse the sperm cell and keep the non-sperm cell intact.

In some embodiment, NaCl or KCl concentration is at least 0.8M, and MgCl₂ concentration is at least 0.25M.

In certain embodiments, the samples are mixed with the first reagent of selective sperm lysis buffer comprising at least one disulfide bond reducing reagent. The second reagent from selective sperm lysis buffer comprising at least one salt reagent is subsequently added to the sample to complete the selective sperm lysis. The second reagent as described herein can be added after 1 minute, 5 minutes, 10 minutes or longer after the first reagent is added. Optionally, the cells in the sample are separated from the first reagent of selective sperm lysis buffer then mixed with the second reagent from selective sperm lysis buffer as described herein.

Based on the teaching, one of the skill in the art can optimize the final concentration of salt and disulfide bond reducing reagent to breakdown the sperm cell while keep the non-sperm cell intact.

In some embodiment of the teaching, the sperm cells and non-sperm cell mixture, wherein the non-sperm cells includes human or mammalian cells selected from, but not limited to, the group consisting of epithelial cells, erythrocytes, platelets, neutrophils, lymphocytes, monocytes, eosinophils, basophils, adipocytes, chondrocytes, tumor cells, neurons, glial cells, astrocytes, red blood cells, white blood cells, macrophages, hair cells, bladder cells, kidney cells, retinal cells, rod cells, cone cells, antigen presenting cells, T cells, B cells, plasma cells, muscle cells, ovarian cells, prostate cells, vaginal epithelial cells, testicular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, osteoclasts, and epithelial cells.

In some embodiment of the teaching, the mixture of cells includes at least sperm cell and epithelial cells.

In certain embodiment of the teaching, the cell mixture includes at least erythrocytes.

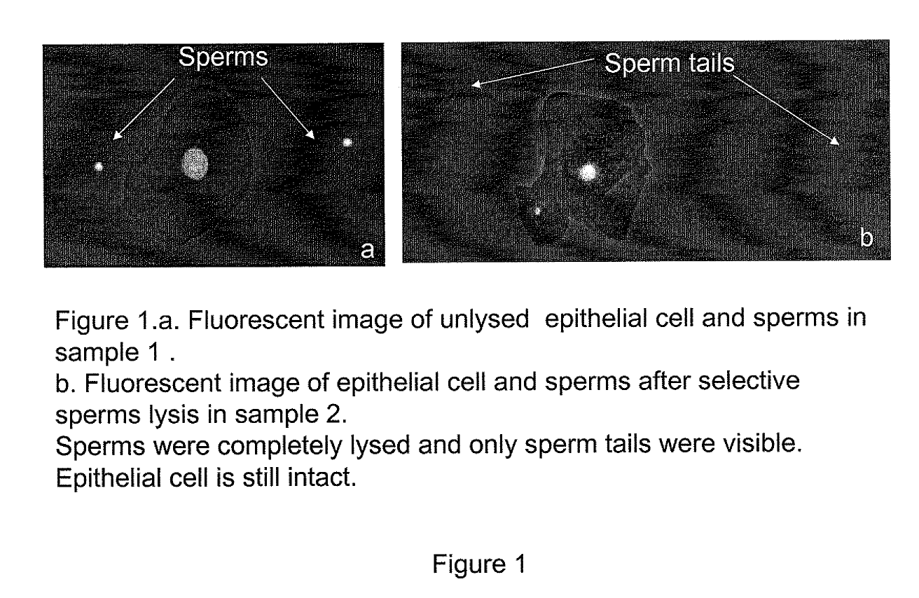

In certain embodiment selective sperm lysis buffer is added to the cell mixture. During an incubation in the buffer selective lysis of sperm occurs, resulting in the release of sperm cell DNA, in the presence of non-sperm cell. (FIG.1).

The incubation is carried out at any temperature and for any length of time that achieves the appropriate results. In certain embodiment, the incubation is carried out at the room temperature for a period of time. Alternatively, the incubation can be carried out at approximately 20-50° C. for about 1 minutes to 4 hours or longer, or 5-10 minutes.

In certain embodiment, the selective cell lysis can be carried out by mixing or pipetting, or gentle mixing and pipetting to avoid mechanical break down of non-sperm cells.

In certain embodiment of the teaching, the cell mixture includes at least sperm cells and epithelial cells, and the sperm cells are selectively lysed in the presence of epithelial cells with a selective sperm lysis buffer comprising at least DTT or any other reagent that breaks disulfide bonds and a salt reagent.

Following lysis, the lysate containing sperm DNA is separated from non-sperm cells and the other debris by separation means. The separation means include, but not limited to, filter that only pass sperm DNA molecule through, filtration, centrifugation means or other liquid phase separtion means.

Optionally, non-sperm cells, epithelial cells are subsequently lysed with conventional buffers or selective epithelial lysis buffer for extraction of epithelial DNA as those described in example 2 to example 5.

In certain embodiment, the selective sperm lysis buffer can be added after non-sperm cells are lysed and DNA of epithelial cells are removed first.

In certain embodiment, the cell mixture includes at least sperm cells and epithelial cells, and the epithelial cells are selectively lysed in the presence of sperm cells with a selective epithelial lysis buffer comprising proteinase K and detergent.

The teaching further comprises the separation of epithelial cell DNA containing lysate and sperm cells by means including, but not limited to filtration or centrifugation means.

The teaching further comprises the lysis of remaining sperm cells with selective sperm lysis buffer and extraction of DNA thereof.

In certain embodiments, DNA can be purified from sperm cells and non-sperm cells. In certain embodiments, DNA can be quantitated, amplified, and STR-typed. In certain embodiment, after the selective extraction of DNA from a particular cell type has been achieved according to the teaching, the DNA can be isolated. DNA isolation can be achieved through a variety of chemical or ionic means. Sambrook et al., Molecular Cloning: A Laboratory Manual. 3rd. ed., Cold Spring Harbor Laboratory Press, Cold Spring Harbor, N.Y., (2001).

Once the DNA has been isolated, various means can be used for DNA typing, such as Restriction Fragment Length Polymorphism (RFLP) Analysis and Polymerase Chain Reaction (PCR)-Based Methods, such as Short Tandem Repeat (STR) analysis and DNA amplification and typing of HLA-DQA1 loci and Polymarker loci.

In some embodiment, DNA can be used for DNA typing. DNA typing refers to the determination of the genetic code variations within a sample, for example using PCR or RFLP, to create a DNA fingerprint.

Polymerase Chain Reaction (PCR) refers to the polymerase chain reaction used to amplify minute amounts of DNA. PCR is a technique in which cycles of denaturation, annealing with primer, and extension with DNA polymerase, are used to amplify the number of copies of a target DNA sequence by >10⁶ times. In general, PCR can be performed according to the protocol described in U.S. Pat. No. 4,683,195. The polymerase chain reaction process for amplifying nucleic acid is further covered by U.S. Pat. Nos. 4,683,195, 4,965,188 and 4,683,202 and European patent Nos. EP 201184 EP 200362.

In some embodiment, DNA samples are subjected to PCR amplification using primers and thermocycling conditions specific for each locus that contains the STR of interest. In particular, A subclass of variable number tandem repeats (VNTRs) is the short tandem repeat (STR), or microsatellite, loci. The STR loci are composed of tandemly repeated sequences, each of which is 2 to 7 bp in length. Loci containing repeat sequences consisting of 4 bp (or tetranucleotides) are used routinely for human identification and, in some cases, 5 bp repeat STRs used. These repeat sequence loci are abundant in the human genome and are highly polymorphic. The number of alleles at a tetranucleotide repeat STR locus ranges usually from 4 to 20. STR loci are amenable to amplification by PCR.

In certain embodiment, the isolated DNA can be used for detection of polymorphic STRs. Individual DNA samples containing amplified alleles can be compared with a size standard such as a DNA marker or locus-specific alletic ladder to determine the alleles present at each locus within the sample. Allelic ladders are constructed for STR loci with the goal of including several or all known alleles with lengths corresponding to amplified fragments containing an integral number of copies of polymorphic sequences. The DNA is then visualized by any number of techniques, including silver staining, radioactive labeling, or fluorescent labeling, various dyes or stains with denaturing or native gel electrophoresis using any available gel matrix or size separation method.

In certain embodiment, the selected amplified alleles are subjected to sequence analysis to confirm the sequence heterogeneity among various alleles.

Further detailed description and examples of DNA amplification and typings are disclosed in Budowle et al. (DNA Typing Protocols: Molecular Biology and Forensic Analysis, Eaton Publishing: Mass., USA 2000)).

### Kits for the Extraction of Sperm DNA

The teaching includes a kit for the separation of sperm and other non-sperm cell DNA that can include (i) reagents for selective lysis of sperm cells Alternately, the kit can include reagents for the further lysis of other cells including, but not limited to epithelial cells. and (ii) an instruction manual to teach the user how to use the kit for the separation of sperm DNA from other cell DNA. The kit can also include filters for lysate and cell separation. The kit can include centrifugation spin column for lysate and cell separation.

In certain embodiment, the kit can be used for separate sperm DNA from non-sperm cells.

In certain embodiment, the kit can include the reagents for lysis of non-sperm cells first, then further with reagent for lysis of sperm cells. The reagent is selective sperm lysis buffer.

In certain embodiments, a selective sperm lysis buffer comprises at least one disulfide bond reducing reagent and at least one salt reagent.

In certain embodiment, a disulfide bond reducing agent selected from reagents including, but not limited to, ME, DTT, GSH and TCEP

In certain embodiments, at least one salt is selected from reagents including but not limited to, LiCl, NaCl, KCl. Li₂SO₄, Na₂SO₄, K₂SO₄, MgCl₂, CaCl₂, MgSO₄ and CaSO₄, NaNO₃, KNO₃, Mg(NO₃)₂, Ca(NO₃)₂. In certain embodiment at least salt comprises NaCl, KCl, or MgCl₂.

In certain embodiment, a selective sperm lysis buffer comprises at least one salt reagent in the concentration at least 0.1M, 0.25M, 0.5M, 1M, 1.5M, 2M or higher.

In certain embodiment, a selective sperm lysis buffer comprises at least one salt reagent in the concentration range from 0.1M to 2M.

In certain embodiments, a selective sperm lysis buffer comprises at least one disulfide bond reducing reagent with concentration ranges from at least 0.01M, 0.05M, 0.1M, 0.2M, 0.3M, 0.4M, 0.5M, 0.7M or 0.8M.

In certain embodiments, a selective sperm lysis buffer comprises disulfide bond reagent selected from the group consisting of ME, DTT, GSH and TCEP and salt reagent selected from the group consisting of NaCd, KCl, MgCl₂ and CaCl₂, wherein the salt concentration is at the concentration to lyse the sperm cell and keep the non-sperm cell intact.

In certain embodiment, NaCl or KCl concentration is at least 0.8M, and MgCl₂ concentration is at least 0.25M.

Based on the teaching, one of the skill in the art can optimize the final concentration of salt and disulfide bond reducing reagent to breakdown the sperm cell while keep the non-sperm cell intact.

Optionally, the kit of the teaching can comprise washing solution for removing extraneous DNA from cells.

The kit of the teaching can be combined or integrated into other applications including, but not limited to, DNA purification.

The teaching is described in further detail in the following examples. These examples are intended to be illustrative only, and are not intended to limit the scope of the teaching.

### EXAMPLES

Aspects of the present teachings can be further understood in light of the following examples, which should not be construed as limiting the scope of the present teachings in any way

### Example 1

### Differential Lysis of Sperm Cells

Two samples were made, each containing about 100K sperm cells and 100K epithelial cells in 60 μl×PBS. 140 μl of 1×PBS and 1 μl of Propidium Iodide (1 mg/mL) were added to the first sample.

100 μl of 2M KCL, 40 μl of 1M DTT and 1 μl of Propidium Iodide (1 mg/mL) were added to the second sample. The final concentrations in the 200 μl second sample were 1 M KCL, and 200 mM DTT. The sample mixture was then mixed and incubated at room temperature for 5 min. Both samples were examined under fluorescent microscopes.

FIG. 1a is the cells image from sample 1, FIG. 1b is the cells image from sample 2. This figure shows that the epithelial cells remained intact in both the first sample and the second sample. The sperm cells, in contrast, were lysed in the second sample, but not in the first sample. Thus, the presence of KCL and DTT in the second sample resulted in selective lysis of the sperm cells in that experiment.

### Example 2

### Differential Extraction Protocol for Swab Samples, Sperm Cell Lysis First

A mock sexual assault swab sample is prepared as follows. Predetermined number of sperm cells is added to a buccal swab containing epithelial cells. The swab is dried at room temperature for 7 days.

The entire swab is placed into a 1.5 ml centrifuge tube and 800 μl of 1× PBS is added. The swab is incubated for 5 minutes in the 1× PBS at room temperature, with occasional agitation using a pipette tip to dissolve any extraneous DNA into the 1× PBS. The centrifuge tube is then spun at 14 k rpm for 2 minutes and the supernatant is discarded. Additional 800 uL of 1×PBS is added to the swab and the swab is agitated using pipette tip to dissolve any extraneous DNA into the 1×PBS. The centrifuge tube is then spun at 14 k rpm for 2 minutes and the supernatant is discarded.

The hundred μl of selective sperm lysis buffer (200 mM DTT, 1M KCl) is then added to the swab. The mixture is incubated for 5 minutes at room temperature, with occasional agitation with a pipette tip to encourage the sperm DNA to dissolve into the selective sperm lysis buffer. The lysate and swab are then transferred to a spin-X filter tube (Corning Incorporated) and spun at 14 k rpm for 5 minutes. The filtrate containing sperm DNA is removed to a new tube and reserved.

Three hundred μl of epithelial cell lysis buffer (AB BloodPrep™ DNA purification solution) is added to the spin-X filter tube containing the swab and incubated for 5 minutes at 70° C. The spin-X filter tube is then spun at 14 k rpm for 5 minutes. The filtrate containing the epithelial DNA is removed to a new tube and reserved.

The sperm DNA and epithelial DNA are each purified using standard DNA purification methods.

### Example 3

### Differential Extraction Protocol for Liquid Samples, Sperm Cell Lysis First

A mock sexual assault liquid sample is prepared as follows. 5K sperm cells are added to 50 ul 1× PBS containing 50K epithelial cells.

The liquid sample is placed in a tube and 500 μl 1× PBS is added. The mixture is then transferred to a spin-X filter tube and then spun at 6 k rpm for 2 minutes to remove any extraneous DNA from the sample. The filtrate is discarded.

One hundred μl of selective sperm lysis buffer (200 mM DTT, 1M KCl) is added to the spin-X tube and the mixture is incubated for 5 minutes at room temperature, with occasional agitation with a pipette tip to encourage the sperm DNA to dissolve into the selective sperm lysis buffer. The spin-X tube is then spun at 8 k rpm for 2 minutes. The filtrate containing sperm DNA is removed to a new tube and reserved.

Three hundred μL of epithelial cell lysis buffer (AB BloodPrep™ DNA purification solution) is added to the spin-X filter tube and incubated for 5 minutes at 70° C. The spin-X filter tube is then spun at 14 k rpm for 5 minutes. The filtrate containing the epithelial DNA is removed to a new tube and reserved.

The sperm DNA and epithelial DNA are each purified using standard DNA purification methods.

### Example 4

### Differential Extraction Protocol for Swab Samples, Epithelial Cell Lysis First

A mock sexual assault swab sample is prepared as described in Example 2. A piece of the swab is placed into a 1.5 ml centrifuge tube and 200 ul selective epithelial cell lysis buffer (2% SDS, 20 mM EDTA, 200 mM NaCL, 20 mM Tris, pH 8, 500 ug/mL protease K) is added. The mixture is incubated at 56° C. for 1 hour. The lysate and swab are transferred to a spin-X filter tube and spun at 14 k rpm for 5 minutes. The filtrate containing epithelial DNA is removed and reserved.

Five hundred μl water is added to the spin-X filter tube. The mixture is agitated with a pipette tip to allow residual epithelial DNA to dissolve in the water. The spin-X filter tube is then spun at 14 k rpm for 5 minutes and the filtrate containing the residual epithelial DNA is discarded.

Three hundred μl of sperm lysis buffer (200 mM DTT, 1M KCl) is added to the spin-X filter tube and the mixture is incubated for 5 minutes at room temperature, with agitation with a pipette to allow the sperm DNA to dissolve in the sperm lysis buffer. The spin-X filter tube is then spun at 14 k rpm for 5 minutes. The filtrate containing the sperm DNA is removed and reserved.

The sperm DNA and epithelial DNA are each purified using standard DNA purification methods.

### Example 5

### Differential Extraction Protocol for Liquid Samples, Epithelial Cell Lysis First

A mock sexual assault liquid sample is prepared as described in Example 3. 100 ul selective epithelial cell lysis buffer (2% SDS, 20 mM EDTA, 200 mM NaCL, 20 mM Tris, pH 8, 500 ug/mL protease K) is added to 100 ul mock sexual assault liquid sample. The mixture is incubated at 56° C. for 1 hour. The lysate is transferred to a spin-X filter tube and spun at 6 k rpm for 2 minutes. The filtrate containing epithelial DNA is removed and reserved.

Five hundred μl water is added to the spin-X filter tube and incubated. The mixture is agitated with a pipette tip to allow residual epithelial DNA to dissolve in the water. The spin-X filter tube is then spun at 6 k rpm for 2 minutes and the filtrate containing the residual epithelial DNA is discarded.

One hundred μl of sperm lysis buffer (200 mM DTT, 1M KCl) is added to the spin-X filter tube and the mixture is incubated for 5 minutes at room temperature, with agitation with a pipette to allow the sperm DNA to dissolve in the sperm lysis buffer. The spin-X filter tube is then spun at 14 k rpm for 5 minutes. The filtrate containing the sperm DNA is removed and reserved.

The sperm DNA and epithelial DNA are each purified using standard DNA purification methods.

### Example 6

### Quantification, Amplification, and STR Typing

Following DNA purification, the sperm DNA is quantitated using the Quantifiler® Y Human Male DNA Quantification Kit (Applied Biosystems, Foster City, Calif.) and the epithelial DNA is quantitated using the Quantifiler® Human DNA Quantification Kit (Applied Biosystems, Foster City, Calif.)

Approximately 1 ng of sperm or epithelial DNA is used for each amplification reaction. STR amplification was carried out using AmpFISTR® Identifiler® PCR Amplification Kit (Applied Biosystems, Foster City, Calif.) on a GeneAmp® PCT System 9700 (Applied Biosystems, Foster City, Calif.) according to the manufacturer's protocol.

STR typing was carried out using ABI PRISM 3100 Genetic Analyzer and data was analyzed using GenMapID3.2.

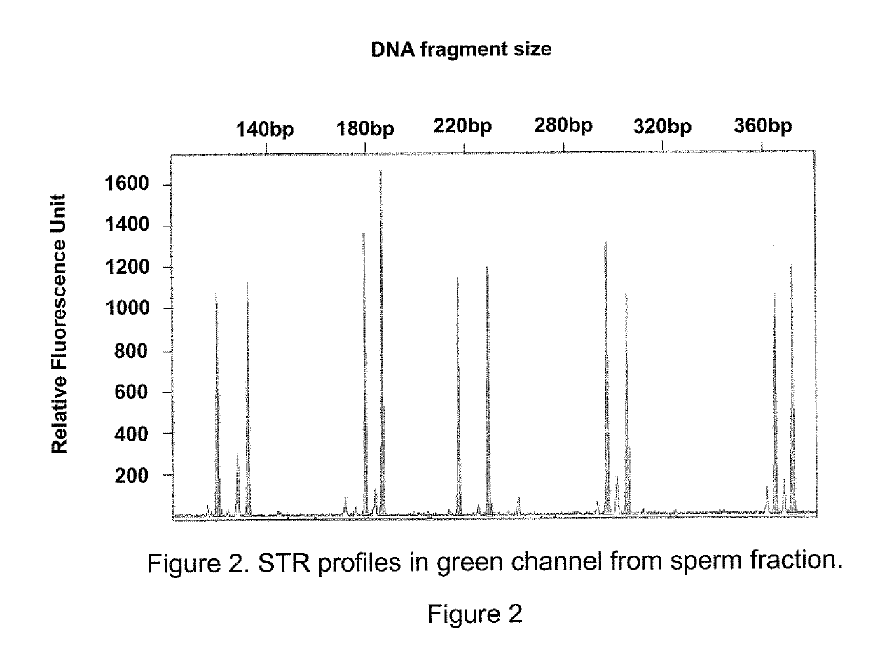

Representative STR analysis results using sperm DNA prepared as described in Example 2 are shown in FIG. 2. The solid peaks are from sperm profile in green channel. The profile is very clean. The same is true for the sperm profiles in other three fluorescent channels. The ratio of sperms to epithelial cells in the mock swab sample is about 1:25.

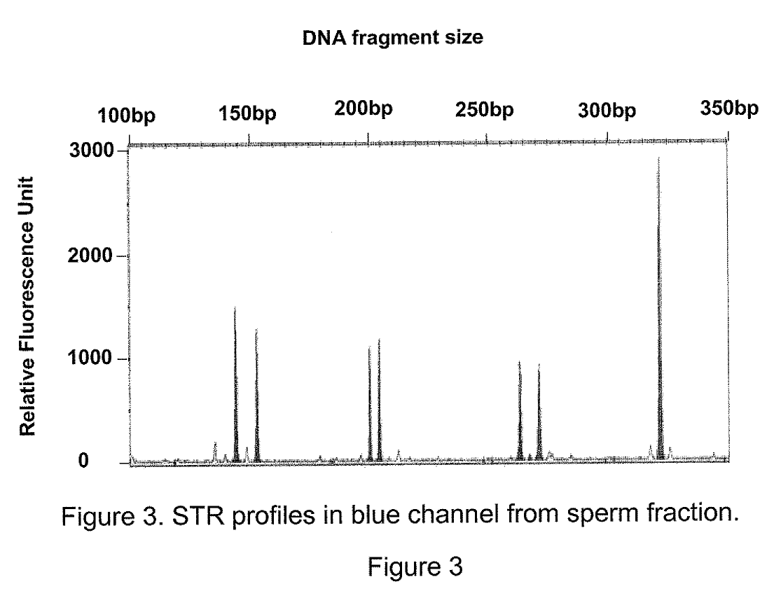

Representative STR analysis results using sperm DNA prepared as described in Example 3 are shown in FIG. 3. The solid peaks are from sperm profile in blue channel. The profile is very clean. The same is true for the sperm profiles in other three fluorescent channels. The ratio of sperms to epithelial cells in the mock sample is about 1:10.

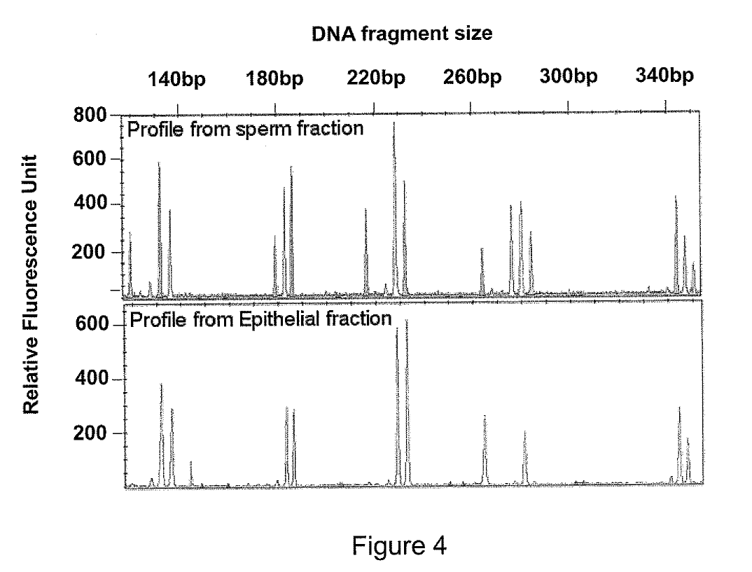

Representative STR analysis results using sperm DNA and epithelial DNA prepared as described in Example 4 are shown in FIG. 4. The top panel is the profile from sperm fraction in green channel. Although a mix profile was obtained, all sperm components can be identified. The same is true for the sperm profiles in other three fluorescent channels. This result is very impressive considering the ratio of sperms to epithelial cells in the sample is about 1:50. The bottom panel is the corresponding profiles from epithelial cell DNA in green channel. The epithelial DNA profile is very clean and no sperm profile is observed. The same is true for the epithelial DNA profiles in other three fluorescent channels.

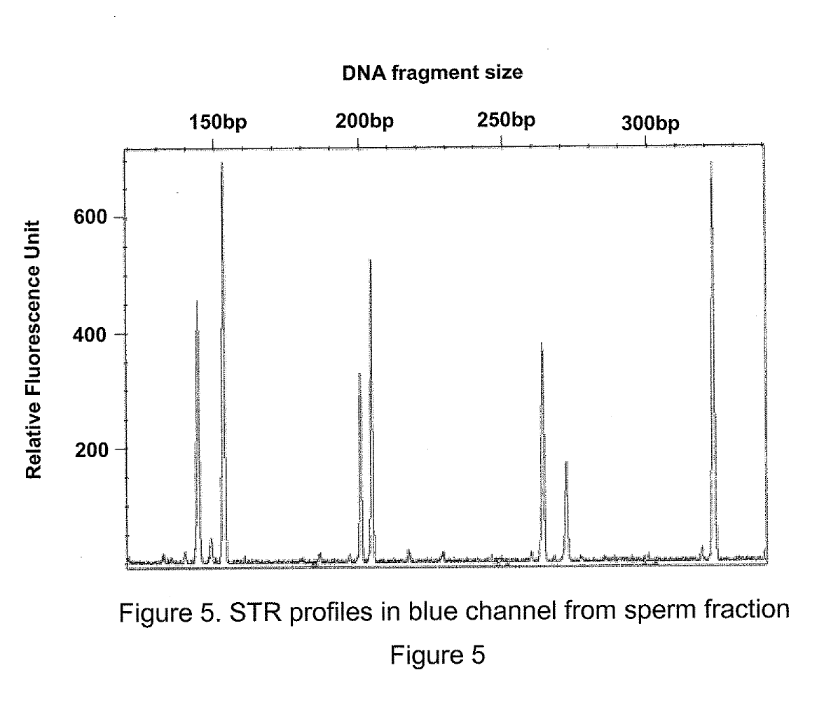

Representative STR analysis results using sperm DNA prepared as described in Example 5 are shown in FIG. 5. Again clean sperm profiles were obtained. The same is true for the sperm profiles in other three fluorescent channels. The ratio of sperms to epithelial cells in the mock sample is about 1:10.

The section headings used above are for organizational purposes only and are not to be construed as limiting the subject matter described in any way.

All literature and similar materials cited in this application, including but not limited to, patents, patent applications, articles, books, treatises, and internet web pages, regardless of the format of such literature and similar materials, are expressly incorporated by reference in their entirety for any purpose.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

I claim:

1. A method of selectively lysing a sperm cell in a mixture of at least one type of non-sperm cell comprises:
i) incubating the mixture with the selective sperm lysis buffer, wherein the buffer comprises at least on disulfide bond reducing reagent and at least one salt reagent, wherein the salt reagent is at a concentration to lyse a sperm cell whereas a non-sperm cell is intact.
ii) lysing the sperm cell, while the non-sperm cell is intact, and
iii) separating the non-sperm cell from the lysed sperm cell.

2. The method of claim 1, wherein at least one type of non-sperm cells comprises erythrocytes, platelets, neutrophils, lymphocytes, monocytes, eosinophils, basophils, adipocytes, chondrocytes, tumor cells, neurons, glial cells, astrocytes, red blood cells, white blood cells, macrophages, hair cells, bladder cells, kidney cells, retinal cells, rod cells, cone cells, antigen presenting cells, T cells, B cells, plasma cells, muscle cells, ovarian cells, prostate cells, vaginal epithelial cells, testicular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, or osteoclasts.

3. The method of claim 1, wherein at least one disulfide bond reducing agent comprises DTT, TCEP, ME or GSH.

4. The method of claim 1, wherein the concentration of at least one salt reagent is at least 0.1M.

5. The method of claim 1, wherein the concentration of at least one salt reagent is at least 0.25M.

6. The method of claim 4, wherein the salt comprises at least LiCl, NaCl, KCl. Li₂SO₄, Na₂SO₄, K₂SO₄, MgCl₂, CaCl₂, MgSO₄, CaSO₄, NaNO₃, KNO₃, Mg(NO₃)₂, or Ca(NO₃)₂.

7. The method of claim 5, wherein the salt comprises at least NaCl, KCl or MgCl₂.

8. A method of differential extraction of sperm cell DNA from a cell mixture from a sample carrier, wherein the cell mixture comprises a sperm cell and at least one type of non-sperm cell, comprises:
i) washing the cell mixture with washing solution and remove the supernatant,
ii) incubating the mixture with the selective sperm lysis buffer, wherein the buffer comprises at least one disulfide bond reducing reagent and at least one salt reagent, wherein the salt reagent is at a concentration to lyse a sperm cell whereas a non-sperm cell is intact.
ii) lysing the sperm cell, while the non-sperm cell is intact, and
iii) separating the non-sperm cell from the lysed sperm cell.

9. The method of claim 8, wherein at least one type of non-sperm cells comprises erythrocytes, platelets, neutrophils, lymphocytes, monocytes, eosinophils, basophils, adipocytes, chondrocytes, tumor cells, neurons, glial cells, astrocytes, red blood cells, white blood cells, macrophages, hair cells, bladder cells, kidney cells, retinal cells, rod cells, cone cells, antigen presenting cells, T cells, B cells, plasma cells, muscle cells, ovarian cells, prostate cells, vaginal epithelial cells, testicular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, or osteoclasts.

10. The method of claim 8, wherein at least one disulfide bond reducing agent comprises DTT, TCEP, ME or GSH.

11. The method of claim 8, wherein the concentration of at least one salt reagent is at least 0.1M.

12. The method of claim 8, wherein the concentration of at least one salt reagent is at least 0.25M.

13. The method of claim 11, wherein the salt comprises at least LiCl, NaCl, KCl. Li₂SO₄, Na₂SO₄, K₂SO₄, MgCl₂, CaCl₂, MgSO₄, CaSO₄, NaNO₃, KNO₃, Mg(NO₃)₂, or Ca(NO₃)₂.

14. The method of claim 12, wherein the salt comprises at least NaCl, KCl or MgCl₂.

15. A kit for isolating sperm DNA from non-sperm cell DNA comprises (i) a selective sperm lysis buffer comprising at least one disulfide bond reducing agent and at least one salt, wherein the salt is at the concentration wherein the sperm cell is lysed while the other cells are intact.

16. The kit of claim 15, where the salt concentration in the selective sperm lysis buffer is at least 0.1M.

17. The kit of claim 16, wherein the salt is comprises at least LiCl, NaCl, KCl. Li₂SO₄, Na₂SO₄, K₂SO₄, MgCl₂, CaCl₂, MgSO₄, CaSO₄, NaNO₃, KNO₃, Mg(NO₃)₂, or Ca(NO₃)₂.

18. The kit of claim 15, wherein the concentration of at least one salt reagent is at least 0.25M.

19. The kit of claim 18, wherein the salt reagent comprises at least NaCl, KCl or MgCl₂.

20. The kit of claim 15, wherein at least one disulfide bond reducing agent comprises at least DTT, TCEP, ME or GSH.

21. The kit of claim 20, where the salt concentration in the selective sperm lysis buffer is at least 0.1M.

22. The kit of claim 21, wherein the salt comprises at least LiCl, NaCl, KCl. Li₂SO₄, Na₂SO₄, K₂SO₄, MgCl₂, CaCl₂, MgSO₄, CaSO₄, NaNO₃, KNO₃, Mg(NO₃)₂, or Ca(NO₃)₂.

23. The kit of claim 20, wherein the concentration of at least one salt reagent is at least 0.5M.

24. The kit of claim 23, wherein the salt reagent comprises at least NaCl, KCl or MgCl₂.
